(19) United States
(12) Patent Application Publication
(10) Pub. No.: US 2018/0369030 A1
(43) Pub. Date: Dec. 27, 2018

(54) FEMININE DISPOSABLE BAG SYSTEM

(71) Applicant: Nasya Myles, Pittsburgh, PA (US)

(72) Inventor: Nasya Myles, Pittsburgh, PA (US)

(21) Appl. No.: 16/020,989

(22) Filed: Jun. 27, 2018

Related U.S. Application Data
(60) Provisional application No. 62/525,468, filed on Jun. 27, 2017.

(51) Int. Cl. A61F 13/551

(52) U.S. Cl. CPC A61F 13/5515; A61F 13/5518; A61F 2013/55195; A61F 2013/55155

(57) ABSTRACT

A disposable bag system including a case member and a plurality of bags with the plurality of bags structured and arranged to be retained within an interior volume of the case member to thereby hold used feminine hygiene products within the interior volume of a bag for disposal.

### CROSS-REFERENCE TO RELATED APPLICATION

The present application is related to and claims priority from prior provisional application Ser. No. 62/525,468, filed Jun. 27, 2017 which application is incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

### 1. FIELD OF THE INVENTION

The present invention relates generally to the field of hygiene products and more specifically relates to a disposable bag system including a case member and a plurality of bags with the plurality of bags structured and arranged to be retained within an interior volume of the case member to thereby hold used feminine hygiene products within the interior volume of a bag for disposal.

### 2. DESCRIPTION OF THE RELATED ART

Feminine hygiene products are personal care products used by women, for menstruation, vaginal discharge, and other bodily functions related to the vulva and vagina. These products are either disposable or reusable. Sanitary, tampons, and pantiliners are disposable feminine hygiene products. Menstrual cups, cloth menstrual pads and period panties are the major categories of reusable feminine hygiene products.

Feminine products are a necessity, however, disposing of them is not always easily carried out in a discreet manner. Unfortunately, used feminine products are left uncovered in homes or public restrooms. To many this can be undesirable.

Ideally, a disposable bag system should be user-friendly and safe in-use and yet would operate reliably and be manufactured at a modest expense. Thus, a need exists for a disposable bag system including a case member and a plurality of bags with the plurality of bags structured and arranged to be retained within an interior volume of the case member to thereby hold used feminine hygiene products within the interior volume of a bag for disposal and to avoid the above-mentioned problems.

### BRIEF SUMMARY OF THE INVENTION

In view of the foregoing disadvantages inherent in the known of hygiene products art, the present invention provides a novel disposable bag system (Entitled the Feminine Disposable Bag System). The general purpose of the present invention, which will be described subsequently in greater detail is to provide a disposable bag system including a case member and a plurality of bags with the plurality of bags structured and arranged to be retained within an interior volume of the case member to thereby hold used feminine hygiene products within the interior volume of a bag for disposal.

A disposable bag system comprising: a case member and a plurality of bags. The case member includes at least one wall member and an interior volume. Wherein one of the at least one wall member having a dispenser aperture therethrough. Wherein the interior volume is adapted to retain and release a plurality of bags therein. Wherein the plurality of bags are adapted to be retained within the interior volume and be individually removed via the dispenser aperture.

The plurality of bags each comprises a flexible material and a drawstring. Wherein the flexible material is formed into a shape of a bag having an opening and an interior volume, and adapted to retain used feminine hygiene products within the interior volume. Wherein the drawstring is movably attached to a periphery of the bag around the opening thereof and adapted to reduce the size of the opening to thereby hold the feminine hygiene products within the interior volume of the bag for disposal. Wherein the plurality of bags are adapted to be retained within the interior volume of the case member and individually removed via the dispenser aperture.

A sanitary napkin system is also disclosed herein comprising: at least one sanitary napkin; a case member; and a plurality of bags. Wherein the at least one sanitary napkin is adapted to be used by females for personal hygiene.

The present invention holds significant improvements and serves as a Disposable Bag System. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, a Disposable Bag System (Entitled the Feminine Disposable Bag System), constructed and operative according to the teachings of the present invention.

FIG. 1 shows a perspective view illustrating a case member and plurality of bags Disposable Bag System according to an embodiment of the present invention.

FIG. 2 is a perspective view illustrating the case member on a keychain of the Disposable Bag System embodiment of the present invention of FIG. 1.

FIG. 3 is a perspective view illustrating a plurality of bags stored inside of case member and individually removed via dispenser aperture 300 according to an embodiment of the present invention of FIG. 1.

FIG. 4 is a perspective view illustrating the bag in an open condition according to an embodiment of the present invention of FIG. 1.

FIG. 5 is a perspective view illustrating user puling closed the bag via a tab located on a drawstring to close the bag shut once used feminine hygiene products are inside to be discarded in a closed condition with used according to an embodiment of the present invention of FIG. 1.

FIG. 6 is a perspective view illustrating a sanitary napkin system having a sanitary napkin; a case member; and a plurality of bags according to an embodiment of the present invention

The various embodiments of the present invention will hereinafter be described in conjunction with the appended drawings, wherein like designations denote like elements.

### DETAILED DESCRIPTION

As discussed above, embodiments of the present invention relate to hygiene products and more particularly to a disposable bag system including a case member and a plurality of bags with the plurality of bags structured and arranged to be retained within an interior volume of the case member to thereby hold used feminine hygiene products within the interior volume of a bag for disposal.

Generally speaking, the disposable bag system comprises a pouch with small scented disposable bags to put used feminine products in for disposal. The scented disposable bags make disposing of feminine waste products more pleasant and convenient. In one embodiment the scented disposable bags are made from an eco-friendly material. A pad and tampon line that is made of eco-friendly de-compostable materials may also be used along with the disposable bags.

The scented disposable bags are opaque such that the used feminine product cannot be seen. Furthermore, each of the scented disposable bags has a drawstring to secure the bag closed once the used feminine product has been placed in the bag. The scented disposable bags are stored in the travel reusable pouch for easy storage and transport. The pouch has a closure that snaps open and closed to access the scented disposable bags. The scented disposable bags are interfolded for easy retrieval from the pouch.

The pouch may include a wrist strap so that the user does not drop the pouch on the dirty bathroom floor. In one embodiment, the pouch is a keychain dispenser and may be half the size of a baby wipes packet. Larger pouches and bags may be produced to replace the paper bags found in toilet stalls.

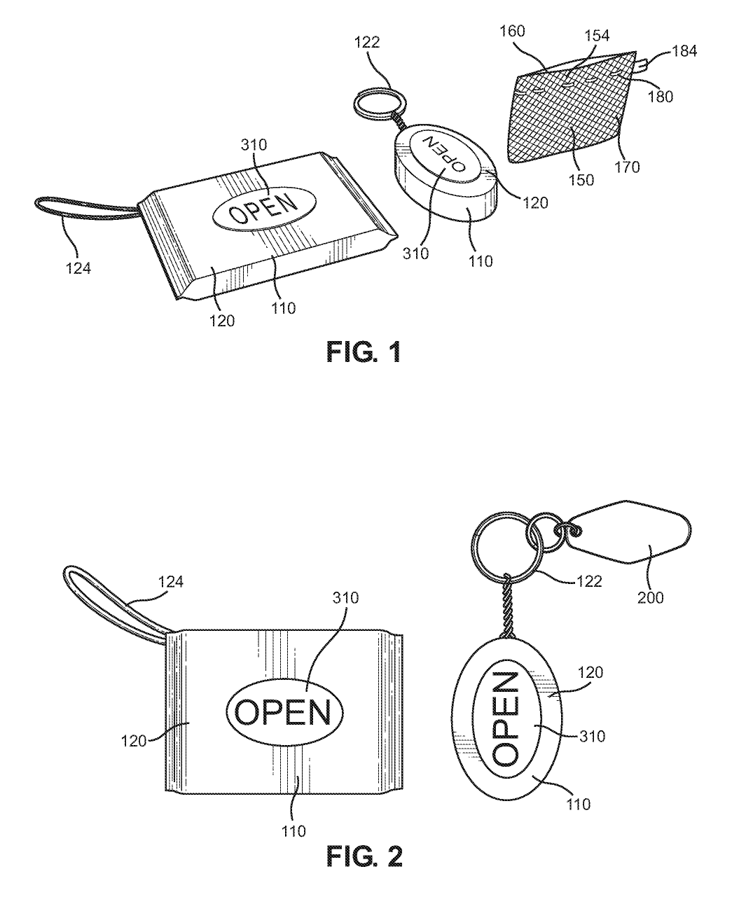

Referring to the drawings by numerals of reference there is shown in FIGS. 1-4, perspective views illustrating disposable bag system 100 according to an embodiment of the present invention.

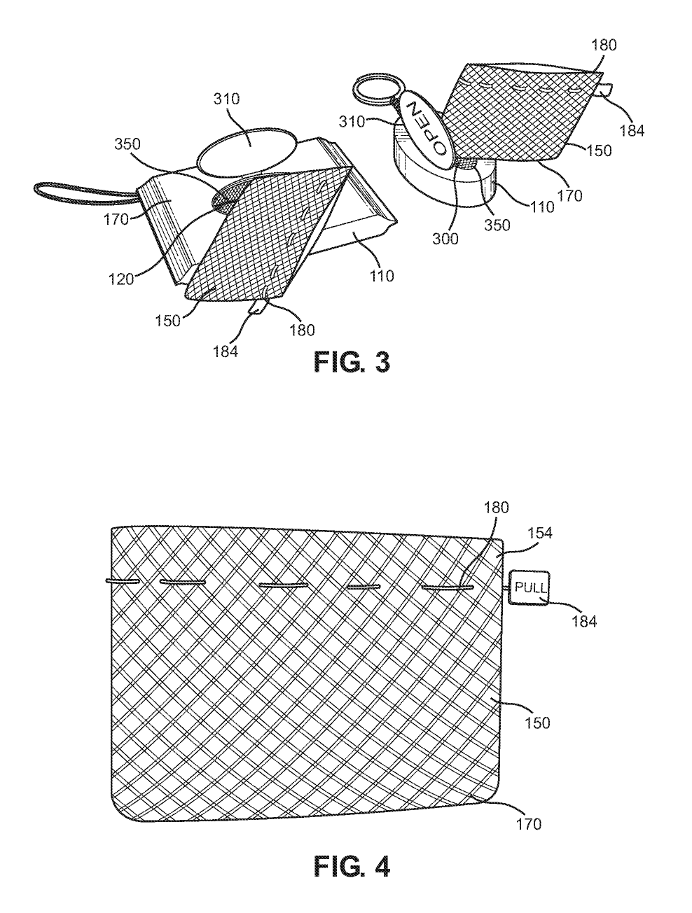

Disposable bag system 100 comprising: case member 110 and plurality of bags 150. Case member 110 includes at least one wall member 120 and interior volume 350. Wherein one of at least one wall member 120 having dispenser aperture 300 therethrough. Wherein interior volume 350 is adapted to retain and release plurality of bags 150 therein as shown in FIG. 3. Wherein plurality of bags 150 are adapted to be retained within interior volume 350 and be individually removed via the dispenser aperture 300.

Wherein case member 110 further includes attachment member 122 on an outer surface of at least one wall member 120 adapted to releasably attach disposable bag system 100 to keychain 200 of FIG. 2 or a belt.

Wherein case member 110 further includes wrist strap 124 attached to an outer surface of at least one wall member 120. Wherein case member 110 further includes panel member 310 adapted to releasably close dispenser aperture 300. Wherein panel member 310 is pivotally connected to an outer surface of at least one wall member 120 to releasably close dispenser aperture 300.

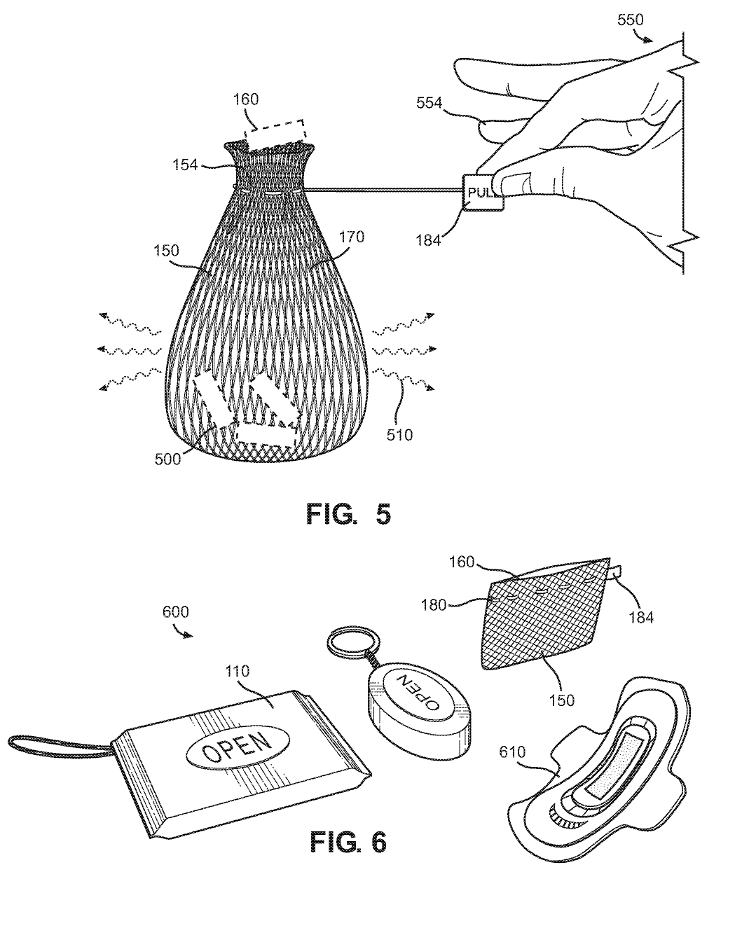

Plurality of bags 150 each comprises flexible material 154 and drawstring 180. Wherein flexible material 154 is formed into a shape of bag 150 having opening 160 and interior volume 170 and adapted to retain used feminine hygiene products 500 within interior volume 170 as shown in FIG. 5. Wherein flexible material 154 is biodegradable. Wherein flexible material 154 is opaque.

Wherein drawstring 180 is movably attached to a periphery of bag 150 around opening 160 thereof and adapted to reduce the size of opening 160 to thereby hold feminine hygiene products 500 within interior volume 170 of bag 150 for disposal as show in FIG. 5. Wherein drawstring 180 is movably threaded into flexible material 154 as shown in FIG. 4. Wherein drawstring 180 includes tab 184 thereon adapted to be grabbed by fingers 554 of user 550 to pull drawstring 180 and reduce the size of opening 160.

Wherein plurality of bags 150 are adapted to be retained within interior volume 350 of case member 110 and individually removed via dispenser aperture 300. Wherein each of plurality of bags 150 is formed having scent 510 adapted to cover up any smell emanating from feminine hygiene products 500 within said interior volume 170 of bag 150.

Referring to the drawings by numerals of reference there is shown in FIG. 6, perspective views illustrating sanitary napkin system 600 according to an embodiment of the present invention.

Sanitary napkin system 600 is also disclosed herein comprising: at least one sanitary napkin 610; case member 110; and plurality of bags 150. Wherein at least one sanitary napkin 600 is adapted to be used by females for personal hygiene.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is:

1. A disposable bag system comprising:
a case member including:
at least one wall member;
wherein one of said at least one wall member having a dispenser aperture therethrough; and
an interior volume;
wherein said interior volume is adapted to retain and release a plurality of bags therein;
wherein said plurality of bags are adapted to be retained within said interior volume and be individually removed via said dispenser aperture; and
a plurality of bags each comprising:
a flexible material;
wherein said flexible material is formed into a shape of a bag having an opening and an interior volume, and adapted to retain used feminine hygiene products within said interior volume; and
a drawstring;
wherein said drawstring is movably attached to a periphery of said bag around said opening thereof and adapted to reduce the size of said opening to thereby hold said used feminine hygiene products within said interior volume of said bag for disposal;
wherein said plurality of bags are adapted to be retained within said interior volume of said case member and individually removed via said dispenser aperture.

2. The disposable bag system of claim 1, wherein said drawstring is movably threaded into said flexible material.

3. The disposable bag system of claim 1, wherein said drawstring includes a tab thereon adapted to be grabbed by the fingers of a user to pull said drawstring and reduce the size of said opening.

4. The disposable bag system of claim 1, wherein said case member further includes an attachment member on an outer surface of said at least one wall member adapted to releasably attach said disposable bag system to a user's keychain or belt.

5. The disposable bag system of claim 1, wherein said case member further includes a wrist strap attached to an outer surface of said at least one wall member.

6. The disposable bag system of claim 1, wherein each of said plurality of bags is formed having a scent adapted to cover up any smell emanating from said used feminine hygiene products within said interior volume of said bag.

7. The disposable bag system of claim 1, wherein said flexible material is biodegradable.

8. The disposable bag system of claim 1, wherein said flexible material is opaque.

9. The disposable bag system of claim 1, wherein said case member further includes a panel member adapted to releasably close said dispenser aperture.

10. The disposable bag system of claim 9, wherein said panel member is pivotally connected to an outer surface of said at least one wall member to releasably close said dispenser aperture.

11. The disposable bag system of claim 1, wherein said plurality of bags are interfolded for easy retrieval from said interior volume of said case member.

12. A sanitary napkin system comprising:
at least one sanitary napkin;
wherein said at least one sanitary napkin is adapted to be used by females for personal hygiene;
a case member including:
at least one wall member;
wherein one of said at least one wall member having a dispenser aperture therethrough; and
an interior volume;
wherein said interior volume is adapted to retain and release a plurality of bags therein;
wherein said plurality of bags are adapted to be retained within said interior volume and be individually removed via said dispenser aperture; and
a plurality of bags each comprising:
a flexible material;
wherein said flexible material is formed into a shape of a bag having an opening and an interior volume, and adapted to retain used feminine hygiene products within said interior volume; and
a drawstring;
wherein said drawstring is movably attached to a periphery of said bag around said opening thereof and adapted to reduce the size of said opening to thereby hold a used one of said at least one sanitary napkin within said interior volume of said bag for disposal;
wherein said plurality of bags are adapted to be retained within said interior volume of said case member and individually removed via said dispenser aperture.

13. The sanitary napkin system of claim 12, wherein said drawstring is movably threaded into said flexible material.

14. The sanitary napkin system of claim 12, wherein said drawstring includes a tab thereon adapted to be grabbed by the fingers of a user to pull said drawstring and reduce the size of said opening.

15. The sanitary napkin system of claim 12, wherein said case member further includes an attachment member on an outer surface of said at least one wall member adapted to releasably attach said disposable bag system to a user's keychain or belt.

16. The sanitary napkin system of claim 12, wherein said case member further includes a wrist strap attached to an outer surface of said at least one wall member.

17. The sanitary napkin system of claim 12, wherein each of said plurality of bags is formed having a scent adapted to cover up any smell emanating from a used one of said at least one sanitary napkin within said interior volume of said bag.

18. The sanitary napkin system of claim 12, wherein said flexible material is biodegradable.

19. The sanitary napkin system of claim 12, wherein said flexible material is opaque.

20. The sanitary napkin system of claim 12, wherein said case member further includes a panel member adapted to releasably close said dispenser aperture; and wherein said panel member is pivotally connected to an outer surface of said at least one wall member to releasably close said dispenser aperture.
